(19) United States
(12) Patent Application Publication
(10) Pub. No.: US 2025/0134925 A1
(43) Pub. Date: May 1, 2025

(54) Bioactive mitochondria encapsulated in a metal organic framework

(71) Applicant: Åbo Akademi, Åbo (FI)

(72) Inventors: Hongbo Zhang, Åbo (FI); Junnian Zhou, Åbo (FI); Chang Liu, Åbo (FI); Yong Guo, Åbo (FI)

(21) Appl. No.: 18/684,198

(22) PCT Filed: Aug. 16, 2022
(86) PCT No.: PCT/FI2022/050533

§ 371 Date: Feb. 16, 2024

(30) Foreign Application Priority Data
Aug. 16, 2021 (FI) 20215857

(51) Int. Cl. A61K 35/12; A61K 47/54; A61K 49/00; G01N 33/50

(52) U.S. Cl. CPC A61K 35/12; A61K 47/547; A61K 49/0004; G01N 33/5079

(57) ABSTRACT

According to an example aspect of the present invention, there is provided isolated bioactive mitochondria coated with a layer of Metal Organic Framework (MOF) and a method for intracellular delivery and release of said coated bioactive mitochondria in cells. The invention also provides a method for maintaining bioactivity and for increasing storage time of bioactive mitochondria by using MOF encapsulation.

### FIELD

The present invention relates to the extraction of cell organelles, in particular mitochondria, to their encapsulation with Metal Organic Frameworks, and to biomedical, pharmaceutical, clinical, medical and research applications of the encapsulated cell organelles.

### BACKGROUND

Mitochondria are one type of organelles-tiny structures that perform specific functions within a cell. All cells in the human body, except for red blood cells, contain one or more, sometimes several thousands, mitochondria. Mitochondria are one of the most important organelles in the cell. The physiological activities involved in the cell include biosynthesis, apoptosis, cell signal transduction (ROS signal, calcium ion, etc.), bioenergy metabolism pathway (aerobic oxidation of glucose, oxidation of fatty acid), Redox state, etc (Bellance et al., 2009).

Increasing evidence (Kroemer and Pouyssegur, 2008) suggests a key role for mitochondrial dysfunction in diabetes, aging, neurodegenerative disorders, and cancers.

As a semi-autonomous organelle, mitochondria are vulnerable to damage from external factors. Changes in mitochondrial function, such as oxidative phosphorylation damage, abnormal energy metabolism, inhibition of apoptosis, autophagy disorders, promotion of immune escape and changes in signal pathways, may affect the occurrence of tumors. Regulating mitochondrial function may prevent the occurrence of tumors.

Based on this, many drugs or small molecule compounds targeting mitochondria have been developed in tumor-targeted therapy. Although they have certain effects, they mainly change one of the many physiological activities involved in mitochondria. Cancer cells also exhibit extensive metabolic rearrangement that makes them more susceptible to alteration of mitochondria than normal cells.

Recent research (Wolf et al., 2019) shows that individual cristae within the same mitochondrion display different membrane potentials and are functionally independent.

In recent studies, it has also been found that there is mitochondrial transfer between different normal cells. The transfer of mitochondria from normal cells to tumor cells has a good effect on inhibiting the physiological activities of tumor cells (Sun et al., 2019). Mitochondria transfer/transplantation is not a totally new concept, but how to do a long-term storage of bioactive mitochondria in this process is challenging.

MOF (Metal-Organic Framework) generally uses metal ions as the connection point and self-assembles with organic ligands to form a crystalline porous material with a periodic network structure. It is not only different from inorganic porous materials, but also different from general organic complexes. Combining the rigidity of inorganic materials and the flexible characteristics of organic materials, it presents huge development potential and attractive development prospects in modern materials research, especially in batteries, materials, catalysis, air purification and biomedicine (Park et al., 2006; Sun et al., 2012).

Encapsulation of biomolecules has been studied for example by Lyu et al (Lyu et al., 2014), Liang et al. (Liang et al., 2015), Einfalt et al. (Einfalt et al., 2018) and Liu et al. (Liu et al., 2019). Patent application publications WO 2016/000032 A1 and WO 2018/000043 A1 also relate to encapsulation of biomolecules in metal organic frameworks. Encapsulation of yeast cells has been studied for example by Liang et al (Liang et al., 2016). However, no studies are available where encapsulation of isolated bioactive mitochondria with Metal Organic Frameworks would have been disclosed or suggested.

In an attempt to storage isolated mitochondria, Vidya N. Nukala et al (Nukala et al., 2006) described a protocol using 10% DMSO combined with freezing. However, this attempt was only partially successful since the isolated mitochondria could be stored only for short time under freezing. Wei Chen et al (2019) disclosed a nanomaterial coating consisting of alternating layers of negative and positive polyelectrolytes (polyacrylic acid and chitosan) applied on isolated mitochondria. The surface-engineered mitochondria showed some respiration competency but required storage at −80° C. In WO 2021/132735 A2, mitochondria with a smaller size were encapsulated by lipid membrane-based vesicles in a microflow channel device. However, in considering that the final nanoparticles after coating are smaller than the size of mitochondria in general, the mitochondria that are bigger than the lipid vesicles might not be coated, or they break to pieces during the coating.

Therefore, there exists a need to improve the storage time of isolated mitochondria by maintaining their bioactivity longer than in the methods of the prior art. This would simplify transportation of the isolated mitochondria and their potential application in therapy and research. Further, there exists a need to provide an efficient method for the intracellular delivery of the isolated mitochondria in a targeted and controlled manner to treat different types of diseases, in particular diseases associated with mitochondrial dysfunction. In addition, there is a need to improve methods of drug screening for targeting active mitochondria.

### SUMMARY OF THE INVENTION

The invention is defined by the features of the independent claims. Some specific embodiments are defined in the dependent claims.

The present invention is based on the finding that freshly obtained isolated mitochondria from healthy cells can be coated, protected, and stored at room temperature by using MOF technology (ZIF-8 etc.) for biomineralization of the mitochondria. Further, the MOF coated isolated mitochondria can be easily transferred into target cells to trigger biological effects including cancer stem cell inhibition.

In its first aspect, the present invention provides isolated bioactive mitochondria coated with a layer of Metal Organic Framework (MOF). In particular, the Metal Organic Frameworks (MOFs) comprise non-cytotoxic MOFs synthesized in an aqueous solution at room temperature and ambient pressure.

According to a second aspect of the present invention, there are provided isolated bioactive mitochondria coated with a layer of Metal Organic Framework (MOF) for use in therapy.

According to a further aspect of the present invention, there are provided isolated bioactive mitochondria coated with a layer of Metal Organic Framework (MOF) for use in intracellular delivery and intracellular release of said mitochondria.

According to another aspect of the present invention, there is provided a method of encapsulating isolated bioactive mitochondria with a coating layer of Metal Organic Framework (MOF), wherein the method comprises the steps of providing MOF precursor compounds, which comprise non-toxic metal ions and organic ligands, and combining in an aqueous solution the mitochondria and the MOF precursor compounds to provide a layer of MOF on the mitochondria.

The invention also provides a method for intracellular delivery and release of isolated bioactive mitochondria in cells, wherein the method comprises (i) providing isolated bioactive mitochondria; (ii) providing Metal Organic Framework (MOF) precursor compounds comprising non-toxic metal ions and non-toxic organic ligands; (iii) coating the isolated bioactive mitochondria with a MOF layer by contacting said MOF precursor compounds with the mitochondria in an aqueous solution; and (iv) incubating the MOF coated mitochondria with the cells to transfect the cells with the MOF coated mitochondria.

The invention also provides use of MOF coated bioactive mitochondria in scientific research models of mitochondria in vitro. This aspect enables studying mitochondria and the unique role it plays in the various mitochondria-associated diseases, including the screening of new signal regulation molecules for mitochondria, the study of interactions between mitochondria and cell nuclei, and the pathology of mitochondria in malignant transformation of cells, degenerative diseases, genetic defect diseases, etc. Other examples of interesting research aspects include but are not limited to the sequence change in the physiological process and the unique role it plays, etc.

According to another aspect of the present invention, there is provided a method of drug screening for targeting active mitochondria in vitro, including drugs that inhibit mitochondrial activity of tumor cells, drugs that inhibit tumor cell energy metabolism, and drugs that activate normal mitochondrial activity for genetic diseases of mitochondrial defects. The advantage of this mitochondria-based screening model is that because there are only mitochondria, there is no interference from the cell nucleus and cross-talk of other signalling pathways, so it is easier to obtain new drugs that specifically target mitochondria with a greater probability

The invention also provides a method of enabling the intracellular and in vivo delivery of live mitochondria, the method comprising coating the isolated bioactive mitochondria with non-toxic Metal Organic Frameworks (MOFs) and transfecting the cells with the MOF coated bioactive mitochondria.

A further aspect of the invention is a method of maintaining bioactivity and improving storage time of isolated bioactive mitochondria, wherein the method comprises coating the isolated bioactive mitochondria with non-toxic Metal Organic Frameworks (MOFs).

Embodiments of the invention comprise MOF coated isolated bioactive mitochondria, which comprise means for stimulus-sensitive release of said mitochondria, wherein said means preferably comprise a stimulus sensitive polymer or other substance in the structure of the MOF-coated biomolecule or stimulus sensitive particles as a template of MOFs. Further, the MOF coated isolated bioactive mitochondria may comprise for example positively charged polymers and/or cell penetrating peptides assembled in between the MOF framework to improve intracellular delivery and release of the isolated bioactive mitochondria.

Considerable advantages are obtained by the invention. First, the MOF encapsulated, freshly isolated, bioactive mitochondria can be stored at room temperature at least 3 weeks, preferably at least 4 weeks under very simple storage conditions of room temperature and normal saline solution. The membrane potential of mitochondria, which is an important functional indicator, remains unchanged, whereas in the currently recognized conventional method and storing on ice, the membrane potential of the mitochondria drops sharply within a few hours.

Second, the MOF systems provide a stable and tight inorganic coating on the isolated bioactive mitochondria, which coating is also resistant to high temperatures, thus further assisting in maintaining and protecting the bioactivity of the mitochondria. In addition, the method of coating mitochondria with MOFs is environmentally friendly and energy efficient and avoids contact with external substances. The obtained MOF coated mitochondria are easy to transport, and the MOF coating does not affect the mitochondrial activity even after long time storage.

Further, the MOF coated isolated bioactive mitochondria can be delivered in a targeted and controlled manner into cells. After endocytosis into the cell, the non-toxic MOF coating degrades under acidic conditions and the mitochondria are released inside the cells. If desired, the MOF coating layer can be modified, for example by adding agents enhancing the encapsulation of the biomolecules, agents improving the intracellular delivery and/or release of the biomolecules inside the cells, agents providing targeted delivery, and any combinations thereof.

Third, the invention provides very good results in inhibiting cancer stem cell population with the MOF coated mitochondria of the invention, which proves that active mitochondria can be successfully delivered to cells by using the methods and products according to the invention. Thus the invention provides means for developing bioactive mitochondria as drugs for all kinds of mitochondria related diseases, while it also finds use in mitochondria related studies.

Further features and advantages of the present technology will appear from the following description of some embodiments.

### BRIEF DESCRIPTION OF THE DRAWINGS

FIG. 1 illustrates TEM characterization of mitochondria@ZIF8 nanoparticles;

FIG. 2 shows confocal microscopy characterization of dual labeled mitrochondria@ZIF8 nanoparticles;

FIG. 3 shows that ZIF-8 coating can maintain mitochondrial membrane potential up to 4 weeks in 0.9% NaCl, RT;

FIG. 4 illustrates FCM analysis which showed the efficiencies of mitochondria@ZIF8 nanoparticles transfer in two cell lines at the indicated time points; and

FIG. 5 illustrates FCM analysis, which confirmed that mitrochondria@ZIF8 nanoparticles transfer into MDA-MB-231 cells can significantly decrease cancer stem cell population (CD44⁺CD24⁻).

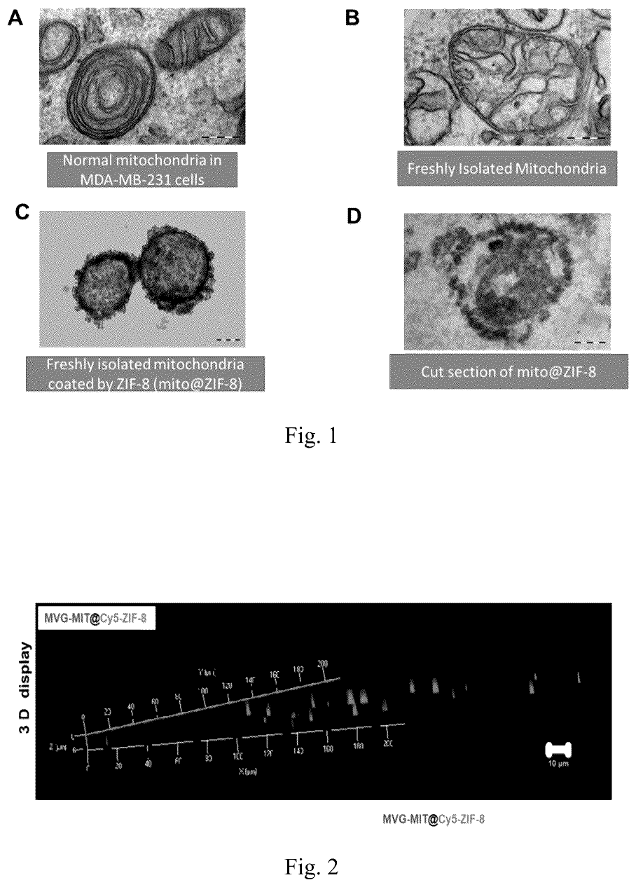

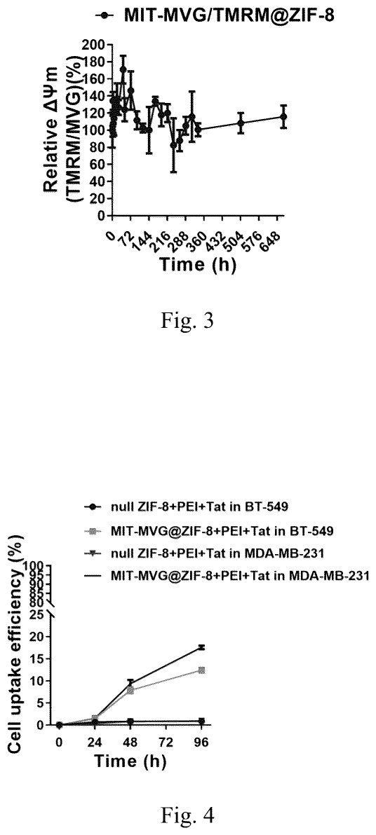

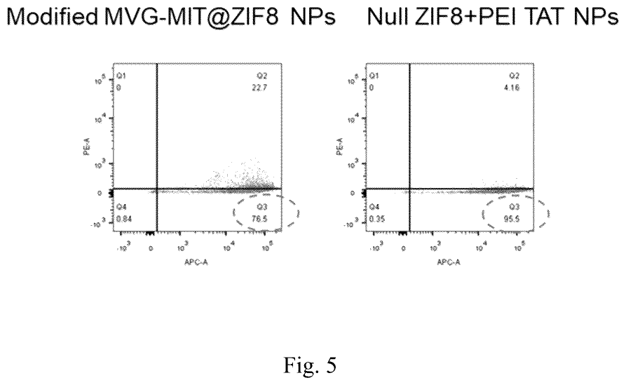

### EMBODIMENTS

### Definitions

“Metal Organic Frameworks” (MOFs) are a class of compounds comprising metal ions or metal clusters coordinated by organic ligands to form one-, two- or three-dimensional structures. In the present context, the term “MOF” relates particularly to metal organic frameworks, which comprise non-toxic metals and non-toxic organic ligands. In particular, the MOFs within this disclosure comprise non-cytotoxic MOFs synthesized in an aqueous solution at room temperature and ambient pressure.

Typically, the “framework” of MOFs is porous, comprising cavities in the form of cages connected by channels. The MOFs may be amorphous or crystalline, typically crystalline. Previously it has been found that it is possible to produce MOFs having a framework that encapsulates a biomolecule, where the biomolecule promotes the formation of the encapsulating framework.

Within this context, “freshly isolated” mitochondria refers to mitochondria isolated from living cells in vitro, by using the methods known by those skilled in the art.

Within this disclosure, the term “bioactive” or “living” in connection with mitochondria refers particularly to mitochondria having a mitochondrial membrane potential of at least 60% of the membrane potential of freshly isolated mitochondria, preferably at least 70%, more preferably at least 80%, still more preferably at least 90% of the membrane potential of freshly isolated mitochondria. Typically, “bioactive” in the context of a substance means that said substance has a biological effect.

As discussed above, the present invention is based on the finding that the freshly isolated bioactive mitochondria, for example freshly isolated healthy breast mitochondria, can be stored at room temperature at least over 3 weeks, even up to 4 weeks or more, after MOF biomineralization.

Thus in one embodiment the invention provides a method for maintaining bioactivity and for improving storage time of isolated bioactive mitochondria, wherein the method comprises coating the isolated bioactive mitochondria with non-toxic Metal Organic Frameworks (MOFs). Typically, bioactivity of freshly isolated bioactive mitochondria is maintained for a storage time of at least 3 weeks, preferably up to 4 weeks or more, at room temperature.

In particular, it was shown that the mitochondrial membrane potential of freshly isolated mitochondria coated for example with one of the widely used MOF types, ZIF-8, can be maintained up to 4 weeks in simple saline solution at RT, while it is dramatically decreased in first 6 h even on ice in mito buffer for the freshly isolated mitochondria. However, although ZIF-8 was the most tested MOF structure for the purposes of the invention, also other types of MOFs that can protect biological macromolecules will also have a similar mitochondrial protective effect. Said MOF structures can also protect mitochondria from cell lysis solution (RIPA) and effects of high temperature.

The Metal Organic Frameworks suitable for use in the present invention are selected from MOFs formed from non-toxic MOF precursors, in particular from non-cytotoxic MOF precursors, such as non-toxic metal ions selected from Ca²⁺, Mg²⁺, Zn²⁺, Fe²⁺, Fe³⁺, Cu²⁺, Eu³⁺, and Zr⁴⁺, and non-toxic organic ligands selected from terephthalates, imidazoles, benzoates, carboxylates and combinations thereof.

Typically, suitable MOFs are selected from zeolitic imidazolate frameworks (ZIFs), preferably from ZIF-8 and ZIF-90, more preferably ZIF-8, other Zn based MOFs, such as IRMOF-3, lanthanide-based MOFs, preferably EuBTC (Eu benzenetricarboxylate frameworks), Fe and/or Al based MOFs, such as MIL-53 and MIL-88B, Cu based MOFs, such as HKUST-1, Zr based MOFs such as UiO-66, UiO-66-NH₂ and UiO-67, and other MOFs comprising non-toxic MOF precursors, such as non-toxic metal ions selected from Ca²⁺, Mg²⁺, Zn²⁺, Fe²⁺, Fe³⁺, Cu²⁺, Eu³⁺, and Zr⁴⁺. Examples of biocompatible MOFs for mitochondria encapsulation and delivery are summarized in the table below.

TABLE 1
Biocompatible MOFs for mitochondria encapsulation and delivery

| Metal | | | Metal | | |
|---|---|---|---|---|---|
| ions | Ligands | Names | ions | Ligands | Names |
| Zn²⁺ | 2-methylimidazole | ZIF-8 | Al²⁺ | 1,4-benzenedicarboxylic acid | MIL-88B |
| | 2-imidazolecarboxaldehyde | ZIF-90 | Cu²⁺ | 1,3,5-benzenetricarboxylic acid | HKUST-1 |
| | 2-amino-1,4-benzenedicarboxylic acid | IRMOF-3 | Zr⁴⁺ | 1,4-benzenedicarboxylic acid | UiO-66 |
| Eu³⁺ | 1,3,5-benzenetricarboxylic acid | EuBTC | | 2-amino-1,4-benzenedicarboxylic acid | UiO-66-NH₂ |
| Fe³⁺ | 1,4-benzenedicarboxylic acid | MIL-53 | | 4,4′biphenyldicarboxylate | UiO-67 |

In an embodiment of the invention, the MOF comprises or is Zeolitic Imidazolate Framework-8 (ZIF-8), formed by coordination between Zn²⁺ ions and 2-methylimidazole (HmIm). Said MOF material has a high surface area, exceptional chemical and thermal stability and negligible cytotoxicity.

As stated above, the MOF coated isolated bioactive mitochondria find use for example in intracellular delivery and intracellular release of said mitochondria in cells, in vitro and in vivo. It is possible to adjust the properties of the MOF structure to improve the intracellular delivery, intracellular release or both.

For improving intracellular delivery and/or intracellular release of mitochondria, the MOF structure may comprise for example interior positively charged polymers, exterior polymers and/or cell penetrating peptides. Examples of positively charged polymers include but are not limited to polyamine polymers, such as polyethyleneimine, while examples of cell penetrating peptides typically include TAT, Penetratin, Polyarginine, P22N, DPV3, DPV6 or combinations thereof.

The intracellular delivery of isolated bioactive mitochondria may find use in any disorders associated with mitochondrial dysfunction, in particular in the treatment of cancer, metabolism related diseases, degenerative diseases, neoplastic diseases, neurodegenerative diseases, neuroimmune disorders, autoimmune diseases, tissue and organ regeneration and repair, aging, and mitochondrial related genetic diseases.

As stated above, it has been found that MOF-coated healthy breast mitochondria exhibit good effects particularly inhibiting cancer stem cell population in breast cancer cells. Based on the importance of mitochondria in the physiological activities of cells, it is reasonable to conclude that this discovery is not limited to the breast cancer system, but the mitochondrial mineralization and transfer strategy of the present invention will work also in other tumor types such as liver cancer, lung cancer, colorectal cancer, prostate cancer, melanoma, leukemia, nasopharyngeal cancer, gastric cancer and other tumor types.

While healthy mitochondria may provide a therapeutic or prophylactic effect on their own, in some embodiments, they may be loaded with therapeutic or prophylactic agents.

In some embodiments of the invention, the release of the mitochondria may be triggered by endogenous stimuli, such as intracellular pH, redox substances, enzymes and/or ATP, preferably by intracellular pH, or by external stimuli selected from light, heat, magnetism and any combinations thereof.

As regards intracellular pH triggered release, it is known that protonation-induced coordination breaking mechanism is one of the most extensive mechanisms for achieving pH-induced drug release. Compared with normal blood and tissue, the pH is lower in tumor microenvironment (pH 5.7-7.8), endosome (pH 5.5-6.0) and lysosome (pH 4.5-5.0). Many acid-sensitive MOFs (such as ZIF-n, MIL-n, UiO-n, and etc.) may be destroyed when being exposed to low pH, resulting in mitochondria release (see e.g. Karakeçili et al, 2019).

pH-sensitive materials are generally dispersed at the structure of the MOF for controlling drug release and preventing early leakage, so it may also be defined as a coating-controlled mitochondria release method. Taking chitosan (CS) as an example, since the pKₐ value of CS is about 6.3, chain protonation of the amino group (—NH₂ become protonated —NH₃+) are caused when the pH is lower than 6.3, which leads to a strong network of intrachain hydrogen bonding, keeping the chains “open” in a swollen stochastic coil conformation and facilitating the degradation of MOF.

Thus the MOF coated mitochondria may comprise means for stimulus-sensitive release of the mitochondria. Said means preferably comprise a stimulus sensitive polymer or other substance in the structure of the MOF-coated mitochondria or stimulus sensitive particle material as a template of MOF. In some embodiments, MOFs comprising stimuli-responsive components may also be called stimuli-responsive MOF hybrids.

Another example of stimulus-responsive release of mitochondria is redox-responsive dissociation of the MOF-coated mitochondria. Compared with normal tissues, the concentration of GSH in tumor tissues is 100-1000-fold higher than that in blood and extracellular matrix. GSH is a strong reducing agent and can be oxidized by oxidizing substances such as disulfide bonds and redox-active substances. Based on these facts, the current mechanisms of GSH-responsive mitochondria carriers based on MOFs mainly involve (1) disulfide bond cleavage mechanism and (2) GSH-sensitive material-mediated mechanism. By introducing the disulfide bond into the ligand of MOF, the disulfide linkage within organic ligands can be cleaved in the presence of GSH, leading to efficient redox-responsive dissociation of MOF and the subsequent release of mitochondria. Redox-active metal ions (such as Mn (IV) and Cu (II) or materials (include MnO2, Cu-MOF) tend to oxidize GSH and lead to the degradation of redox-responsive materials.

In some embodiments, the release of the mitochondria may be triggered by ATP-responsive release. Adenosine triphosphate (ATP), an unstable high-energy compound widely present in living organisms, owns strong coordination ability with some metals ions because of the abundant lone-pair electron of N. Using ATP to compete with the metal sites in MOF for competition coordination will initiate the cleavage of the MOF framework. The competition coordination between ATP and Zn2+ of ZIF-90 is most extensively studied in MOF-based drug delivery.

In some embodiments the MOF coated mitochondria comprise means for release of the biomolecule by light, heat, magnetism or any combinations thereof, wherein said means preferably comprise a thermosensitive polymer layer on the MOF coated mitochondria or thermosensitive material as a template of MOFs.

The main mechanism of light-controlled mitochondria release is to control the mitochondria release by conformational changes, chemical bond cleavage or photothermal conversion of the molecules/materials under illumination. The ligand design strategy is also suitable for designing light-responsive MOF by selecting some special light-sensitive molecules as ligands. Photo-responsive MOFs, such as porphyrin MOFs and UiO-AZB, are often obtained by designing some reactive oxygen species or photoactive molecules (such as porphyrin, azobenzenedicarboxylate (AZB), anthracene and its derivatives, indocyanine green (ICG)) into the structure of MOF. For example, experimental results have shown that AZB is degraded into constituent ions (AZB²⁻ and Zr⁴⁺) under UV illumination, which caused the cleavage of the MOF structure, eventually leading to drug release (Roth Stefaniak et al, 2018).

In some embodiments, the MOF coated mitochondria comprise means for thermal-responsive release of the mitochondria, wherein said means preferably comprise a thermosensitive polymer layer on the MOF coated mitochondria or thermosensitive material as a template of MOFs. Higher temperature tends to decline the stability of the MOF structure in the presence of the polymer. The phase transition of the polymer at high temperatures can enhance the drug release rate.

The method of encapsulating isolated bioactive mitochondria with a coating layer of Metal Organic Framework (MOF) typically comprises the step of: (i) providing MOF precursor compounds, which comprise non-toxic metal ions and organic ligands; and (ii) combining in an aqueous solution the mitochondria and MOF precursor compounds to provide a layer of MOF on the mitochondria.

In particular, the method of encapsulating isolated bioactive with a coating layer of Metal Organic Framework (MOF) comprises the steps of: (i) providing two kinds of MOF precursor aqueous solutions, which comprise non-cytotoxic metal ions solution and non-cytotoxic organic ligands solution; (ii) dispersing fresh isolated mitochondria into the organic ligands aqueous solution for pre-incubation, thereby attaching negatively charged organic ligands to the surface of positively charged mitochondrial membranes by electrostatic interaction; and (iii) combining the metal ions aqueous solution with the mitochondria/organic ligands aqueous solutions to provide a layer of MOF on the mitochondria.

The mitochondria may be coated in a very short time in aqueous conditions to keep the bioactivity best. Once the mitochondria and an aqueous precursor solution are mixed, the MOF layer of encapsulated mitochondria is formed immediately and then aged, typically for 10 minutes, to obtain the nanomaterial, i.e. the MOF coated mitochondria.

If desired, substances or agents, which enhance the intracellular delivery, and/or intracellular release of the mitochondria, may be added into the aqueous solution, whereby said agents are assembled in the MOF structure on top of the mitochondria.

As stated above, said agents or substances may be selected for example from positively charged polymers (such as polyamine polymers, preferably polyethyleneimine and PLGA-PEG/G0-C14), cell penetrating peptides, including TAT, Penetratin, Polyarginine, P22N, DPV3, DPV6 or combinations thereof.

The present invention thus enables a method for the intracellular and in vivo delivery of bioactive mitochondria, wherein the method comprises coating the isolated bioactive mitochondria with non-toxic Metal Organic Frameworks (MOFs) and transfecting the cells with the MOF coated bioactive mitochondria.

Moreover, the present invention provides for the first time a solution to the problem how to store isolated bioactive mitochondria longer than few hours so that their bioactivity is maintained during storage.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

### EXPERIMENTAL

### Example 1: ZIF-8 Encapsulation for Freshly Isolated Mitochondria and Cell Uptake Assay

One day prior to isolation, stain mitochondria in live cells with a final concentration of 25 nM Mito View™ Green (Biotium) at 37 degree protected from light, according to the manufacture's protocol for visualizing the coated mitochondria in the target cells.

Then test whether the cells are clearly stained under fluorescent microscope. Ten dish of MCF-10A cells (reach 80%-90%) to digest for 20 min at 37 degree in cell incubator and cell counting.

Immediately before use, add protease inhibitors to Reagent A (800 ul in one 1.5 mL EP tube) and Reagent C (800 ul+500 ul, in two 1.5 mL EP tube, respectively); only add inhibitors to the reagent amount being used for the procedure and not to the stock solutions, according to the manufacture's protocol (Mitochondria Isolation Kit for Cultured Cells, Invitrogen). Needs ice bath.

Pellet 2×10⁷ cells by centrifuging harvested cell suspension in a 15 mL microcentrifuge tube (needs adaptor) at ˜850×g for 2 minutes (using high speed centrifuge). Carefully remove and discard the supernatant. And set up high speed centrifuge into 4 degree.

Immediately before use, add protease inhibitors to Reagent A (800 ul in one 1.5 mL EP tube) and add into the cells in 15 mL microcentrifuge tube, Vortex at medium speed for 5 seconds and incubate tube on ice for exactly 2 minutes. Note: Do not exceed the 2 minute-incubation.

Incubate tube on ice for 5 minutes, vortex at maximum speed every minute.

Immediately before use, add 8 μL protease inhibitors to 800 μL Reagent C (in 1.5 mL EP tube) and add them in cell-contained tube. Invert tube several times to mix (do not vortex).

Centrifuge tube at 700×g for 10 minutes at 4° C. (using high speed centrifuge).

Transfer the supernatant to a new, 15 mL tube and centrifuge at 3000×g for 15 minutes at 4° C. to obtain a more purified fraction of mitochondria, with >50% reduction of lysosomal and peroxisomal contaminants.

Transfer the supernatant (cytosol fraction) to a new tube. The pellet contains the isolated mitochondria.

Immediately before use, add 5 μL protease inhibitors to 500 μL Mitochondria Isolation Reagent C to the pellet, and centrifuge at 12,000×g for 5 minutes. Discard the supernatant.

Then mitochondria pellets were added into a solution of 2-methylimidazole at 160 mM, followed by dropping zinc acetate solutions (40 mM) into mitochondria-contained 2-methylimidazole solutions, gently mix them and leave it for at least 10 minutes in RT; To increase the efficiency of cell uptake, it can be modified by PEI and cell penetrating peptide Tat.

Centrifuge at 13000 rpm, 5 min, discard the supernatant. Then wash the MIT@ZIF8 nanoparticles with 0.9% NaCl solution for twice. Take one small part of the samples for characterization by TEM.

For biology effects investigation, 10-fold dilution of NPs and added into medium of cells in series of dilutions for at least 48 hours.

### Example 2: Flow Cytometry Analysis

After transfecting the cells with the nanoparticles for 48 hours, they were digested with trypsin (Invitrogen) to a single cell suspension, washed twice with PBS, resuspended in 800 ul PBS, and tested on the machine. During the test, the Alex 488/FITC channel was used for analysis. Cells under the same conditions transfected with empty nanoparticles without mitochondria served as a control group. The instrument is Zeiss LSM780 (Turku Bioscience center, Turku, Finland).

### Example 3: TEM Analysis

The nanoparticles obtained by centrifugation are further diluted 10-20 times with deionized water on the basis of the original volume, and 1-2 drops are added to the special copper grids for TEM. After air drying for 24 hours, the samples are tested by transmission electron microscopy. Transmission electron microscopy (TEM) was performed on a JEOL JEM-1400Plus electron microscope operated at 80 kV.

### Example 4: Mitochondrial Membrane Potential

The mitochondrial membrane potential (ΔΨm) generated by proton pumps (Complexes I, III and IV) is an essential component in the process of energy storage during oxidative phosphorylation. Together with the proton gradient (ΔpH), ΔΨm forms the transmembrane potential of hydrogen ions which is harnessed to make ATP.

In the present experiments, Tetramethylrhodamine methyl ester perchlorate (TMRM) assay kit was used to detect the mitochondrial membrane potential.

Healthy mitochondrial membranes maintain a difference in electrical potential between the interior and exterior of the organelle, referred to as a membrane potential. Tetramethylrhodamine, methyl ester (TMRM) is a cell-permeant dye that accumulates in active mitochondria with intact membrane potentials. If the cells are healthy and have functioning mitochondria, the signal is bright. Upon loss of the mitochondrial membrane potential, TMRM accumulation ceases and the signal dims or disappears. TMRM signal can be detected with fluorescence microscopy, flow cytometry, cell sorting, high throughput screening, and high content analysis.

In brief, image-iT™ TMRM Reagent (Cat. No. 134361) is provided as a 1000× concentrated stock solution at a concentration of 100 μM in DMSO. To use it, simply dilute the stock solution 1000× in cell growth or imaging medium. For detection of TMRM signal, use 488 nm laser for excitation and a 570±10 nm emission filter for detection.

Mito View™ dyes are fluorogenic stains for staining mitochondria in live cells. The dyes are membrane permeable and become brightly fluorescent upon accumulation in the mitochondrial membrane. MitoView™ Green staining is not dependent on membrane potential, and can be used to label all the mitochondria. For detection of Mito View™ signal, use 490 nm laser for excitation and a 523 nm emission filter for detection.

We perform double staining of TMRM and MVG in living cells before extracting mitochondria, and then extract mitochondria, a part of which is used for mineralization, and the non-mineralized mitochondria are used as control.

The signal is read by a fluorescence spectrophotometer. The relative mitochondrial membrane potential is equal to TMRM fluorescence signal/MVG fluorescence signal.

### Results

From the results it can be seen that freshly obtained isolated mitochondria can be coated by ZIF-8. ZIF-8 coating efficiency was around 72%.

Moreover, MIT@ZIF-8 NPs can be released up to around 70% at 6 h in pH 5.0 rather than in pH 7.4.

It was found that the mitochondrial membrane potential of MIT@ZIF-8 can be maintained up to 4 weeks in simple saline solution at RT, while it is dramatically decreased in first 6 h even on ice in mito buffer for the freshly isolated mitochondria; ZIF-8 can also protect mitochondria from cell lysis solution (RIPA) and high temperature destroy.

Finally, modified MIT@ZIF-8 NPs by PEI and Tat can optimize the characteristics including size, surface charge, and aqueous dispersion. Uptake efficiency is 12-17% in BT-549 and MDA-MB-231 cells, of which some transplanted MIT merged with the endogenous mitochondria. A significant decrease in the cell growth and CD44 CD24 population was detected, although the cell transfection efficiency after PEI and Tat cell penetrating peptide modification was 10-20%. In the malignant phenotype breast cancer cells (MDA-MB-231), the proportion of down-regulated CSC in cancer cells was about 20%.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

### INDUSTRIAL APPLICABILITY

At least some embodiments of the present invention find industrial application in pharmaceutical and diagnostic industry, including pharmaceutical testing, personal medicine, tissue engineering and remodeling.

ACRONYMS LIST

| | |
|---|---|
| CSC | cancer stem cells |
| DMSO | Dimethyl sulfoxide |
| MIT | mitochondria |
| MOF | Metal organic framework |
| NPs | nanoparticles |
| PEI | Polyethyleneimine |
| RIPA | Radioimmunoprecipitation assay buffer |
| ROS | reactive oxygen species |
| RT | Room temperature |
| Tat | Trans-activator of tranion |
| TEM | Transmission electron microscope |
| ZIFs | Zeolitic imidazolate frameworks |

### CITATION LIST

### Patent Literature

WO 2016/000032 A1
WO 2018/000043 A1
WO 2021/132735 A2

### Non Patent Literature

Bellance, N., Lestienne, P., and Rossignol, R. (2009). Mitochondria: from bioenergetics to the metabolic regulation of carcinogenesis. Front Biosci (Landmark Ed) 14, 4015-4034.
Chen, W., Shi, K., Chu, B., Wei, X., and Qian, Z. (2019). Mitochondrial Surface Engineering for Multidrug Resistance Reversal. Nano Lett. 2019, 19, 2905-2913.
Einfalt, T., Witzigmann, D., Edlinger, C., Sieber, S., Goers, R., Najer, A., Spulber, M., Onaca-Fischer, O., Huwyler, J., and Palivan, C. G. (2018). Biomimetic artificial organelles with in vitro and in vivo activity triggered by reduction in microenvironment. Nat Commun 9, 1127.
Karakeçili, A., Topuz, B., Korpayev, S., Erdek, M. (2019). Metal-organic frameworks for on-demand pH controlled delivery of vancomycin from chitosan scaffolds. Materials Science & Engineering C 105 (2019) 110098
Kroemer, G., and Pouyssegur, J. (2008). Tumor cell metabolism: cancer's Achilles' heel. Cancer Cell 13, 472-482.
Liang, K., Ricco, R., Doherty, C. M., Styles, M. J., Bell, S., Kirby, N., Mudie, S., Haylock, D., Hill, A. J., Doonan, C. J., et al. (2015). Biomimetic mineralization of metal-organic frameworks as protective coatings for biomacromolecules. Nat Commun 6, 7240.
Liang, K., Richardson, J. J., Cui, J., Caruso, F., Doonan, C. J., and Falcaro, P. (2016). Metal-Organic Framework Coatings as Cytoprotective Exoskeletons for Living Cells. Adv Mater 28, 7910-7914.
Liu, J., Guo, Z., and Liang, K. (2019). Biocatalytic Metal-Organic Framework-Based Artificial Cells. Advanced Functional Materials 29, 1905321.
Lyu, F., Zhang, Y., Zare, R. N., Ge, J., and Liu, Z. (2014). One-pot synthesis of protein-embedded metal-organic frameworks with enhanced biological activities. Nano Lett 14, 5761-5765.
Nukala, V. N., Singh, I. N., Davis, L. M., and Sullivan, P. G. (2006). Cryopreservation of brain mitochondria: a novel methodology for functional studies. J Neurosci Methods 152, 48-54.
Park, K. S., Ni, Z., Cote, A. P., Choi, J. Y., Huang, R., Uribe-Romo, F. J., Chae, H. K., O'Keeffe, M., and Yaghi, O. M. (2006). Exceptional chemical and thermal stability of zeolitic imidazolate frameworks. Proc Natl Acad Sci USA 103, 10186-10191.
Roth-Stefaniak, K., Epley, C. C., Novak, J. J., McAndrew, M. L., Cornell, H. D., Zhu, J., McDaniel, D. K., Davis, J. L., Allen, I. C., Morris, A. J. and Grove, T. Z. (2018). Photo-triggered release of 5-fluorouracil from a MOF drug delivery vehicle. Chem. Commun., 2018, 54, 7617-7620.
Sun, C., Liu, X., Wang, B., Wang, Z., Liu, Y., Di, C., Si, J., Li, H., Wu, Q., Xu, D., et al. (2019). Endocytosis-mediated mitochondrial transplantation: Transferring normal human astrocytic mitochondria into glioma cells rescues aerobic respiration and enhances radiosensitivity. Theranostics 9, 3595-3607.
Sun, C. Y., Qin, C., Wang, X. L., Yang, G. S., Shao, K. Z., Lan, Y. Q., Su, Z. M., Huang, P., Wang, C. G., and Wang, E. B. (2012). Zeolitic Imidazolate framework-8 as efficient pH-sensitive drug delivery vehicle. Dalton Trans 41, 6906-6909.
Wolf, D. M., Segawa, M., Kondadi, A. K., Anand, R., Bailey, S. T., Reichert, A. S., van der Bliek, A. M., Shackelford, D. B., Liesa, M., and Shirihai, O. S. (2019). Individual cristae within the same mitochondrion display different membrane potentials and are functionally independent. EMBO J 38, e101056.

1. Isolated bioactive mitochondria coated with a layer of Metal Organic Framework (MOF).

2. Isolated bioactive mitochondria coated with a layer of Metal Organic Framework (MOF) for use in therapy.

3. Isolated bioactive mitochondria coated with a layer of Metal Organic Framework (MOF) for use in intracellular delivery and intracellular release of said mitochondria.

4. The isolated bioactive mitochondria for use according to claim 3, wherein the MOF layer comprises interior positively charged polymers, exterior polymers and/or cell penetrating peptides to improve the intracellular delivery, intracellular release or both.

5. The isolated bioactive mitochondria for use according to claim 4, wherein said positively charged polymers are selected from polyamine polymers, such as polyethyleneimine, and the cell penetrating peptides are selected from TAT, Penetratin, Polyarginine, P22N, DPV3, DPV6 and combinations thereof.

6. The mitochondria for use according to any one of claims 3 to 5, wherein the mitochondria provide a therapeutic or prophylactic effect and/or are loaded with therapeutic or prophylactic agents.

7. The mitochondria for use according to any one of claims 3 to 6 wherein the release of the mitochondria is triggered by endogenous stimuli, such as intracellular pH, redox substances, enzymes and/or ATP, preferably by intracellular pH, or by external stimuli selected from light, heat, magnetism and any combinations thereof.

8. The mitochondria for use according to any one of claims 3 to 7, wherein the MOF coated mitochondria comprise means for stimulus-sensitive release of the mitochondria, wherein said means preferably comprise a stimulus sensitive polymer or other substance in the structure of the MOF-coated mitochondria or stimulus sensitive particle material as a template of MOFs.

9. The mitochondria for use according to any one of claims 3 to 8, wherein the MOF coated mitochondria comprise means for release of the mitochondria by light, heat, magnetism or any combinations thereof, wherein said means preferably comprise a thermosensitive polymer layer on the MOF coated mitochondria or thermosensitive material as a template of MOFs.

10. The mitochondria for use according to any one of claims 3 to 9 in disorders associated with mitochondrial dysfunction, in particular in the treatment of cancer, metabolism related diseases, degenerative diseases, neoplastic diseases, neurodegenerative diseases, neuroimmune disorders, autoimmune diseases, tissue and organ regeneration and repair, aging, and mitochondrial related genetic diseases.

11. The mitochondria for use according to claim 10 in the treatment of cancer, in particular breast cancer, liver cancer, lung cancer, colorectal cancer, prostate cancer, melanoma, leukemia, nasopharyngeal cancer and gastric cancer.

12. The mitochondria according to any one of the preceding claims, wherein the Metal Organic Framework is selected from MOFs formed from non-toxic MOF precursors, such as non-toxic metal ions selected from Ca²⁺, Mg²⁺, Zn²⁺, Fe²⁺, Fe³⁺, Cu²⁺, Eu³⁺, and Zr⁴⁺, and non-toxic organic ligands selected from terephthalates, imidazoles, benzoates, carboxylates and combinations thereof.

13. The mitochondria according to any one of the preceding claims, wherein the Metal Organic Framework is selected from zinc imidazolate frameworks (ZIFs), preferably from ZIF-8 and ZIF-90, more preferably ZIF-8, other Zn based MOFs, such as IRMOF-3, lanthanide-based MOFs, preferably EuBTC (Eu benzenetricarboxylate frameworks), Fe and/or Al based MOFs, such as MIL-53 and MIL-88B, Cu based MOFs, such as HKUST-1, Zr based MOFs such as UiO-66, UiO-66-NH₂ and UiO-67, and other MOFs comprising non-toxic MOF precursors, such as non-toxic metal ions selected from Ca²⁺, Mg²⁺, Zn²⁺, Fe²⁺, Fe³⁺, Cu²⁺, Eu³⁺, and Zr⁴⁺.

14. The mitochondria according to any claim 12 or 13, wherein the non-cytotoxic metal ions comprise Zn²⁺ and the non-toxic organic ligands comprise imidazole, preferably 2-methylimidazole.

15. The isolated bioactive mitochondria according to claim 1 or the isolated bioactive mitochondria for use according to any one of claims 2 to 14, coated with a layer of non-cytotoxic Metal Organic Framework (MOF) synthesized in aqueous solution at room temperature and ambient pressure.

16. A method of encapsulating isolated bioactive mitochondria with a coating layer of Metal Organic Framework (MOF), the method comprising:
providing MOF precursor compounds, which comprise non-toxic metal ions and organic ligands;
combining in an aqueous solution the mitochondria and MOF precursor compounds to provide a layer of MOF on the mitochondria.

17. The method according to claim 16, comprising the steps of:
providing two kinds of MOF precursor aqueous solutions, which comprise non-cytotoxic metal ions solution and non-cytotoxic organic ligands solution;
dispersing fresh isolated mitochondria into the organic ligands aqueous solution for pre-incubation, thereby attaching negatively charged organic ligands to the surface of positively charged mitochondrial membranes by electrostatic interaction; and
combining metal ions aqueous solution with the mitochondria/organic ligands aqueous solution to provide a layer of MOF on the mitochondria.

18. A method for intracellular delivery and release of isolated bioactive mitochondria in cells, comprising the steps of:
providing isolated bioactive mitochondria;
providing Metal Organic Framework (MOF) precursor compounds comprising non-toxic metal ions and non-toxic organic ligands;
coating the isolated bioactive mitochondria with a MOF layer by contacting said MOF precursor compounds with the mitochondria in an aqueous solution;
incubating the MOF coated mitochondria with the cells to transfect the cells with the MOF coated mitochondria.

19. The method according to any one of claims 16 to 18, wherein the method comprises a step of adding agents, which enhance the intracellular delivery, intracellular release or both of the mitochondria, into the aqueous solution, whereby said agents are assembled in the MOF structure.

20. The method according to claim 19, wherein said agents are selected from positively charged polymers (such as polyamine polymers, preferably polyethyleneimine and PLGA-PEG/G0-C14), cell penetrating peptides, including TAT, Penetratin, Polyarginine, P22N, DPV3, DPV6 or combinations thereof.

21. The method according to any one of claims 16 to 20, wherein the non-toxic metal ions are selected from Ca²⁺, Mg²⁺, Zn²⁺, Fe²⁺, Fe³⁺, Cu²⁺, Eu³⁺, and Zr⁴⁺, and the non-toxic organic ligands are selected from terephthalates, imidazoles, benzoates and carboxylates.

22. The method according to any one of claims 16 to 21, wherein the non-toxic metal ions comprise Zn²⁺ and the non-toxic organic ligands comprise imidazole, preferably 2-methylimidazole.

23. Use of non-cytotoxic Metal Organic Frameworks (MOFs) for storage of isolated bioactive mitochondria.

24. A method of maintaining bioactivity and for improving storage time of isolated bioactive mitochondria, wherein the method comprises coating the isolated bioactive mitochondria with non-cytotoxic Metal Organic Frameworks (MOFs).

25. The method according to claim 24, wherein bioactivity of freshly isolated bioactive mitochondria is maintained for a storage time of at least 3 weeks, preferably up to 4 weeks, more preferably for at least 4 weeks, at room temperature.

26. Use of MOF coated bioactive mitochondria in research models of mitochondria in vitro.

27. A method of drug screening for targeting active mitochondria in vitro, wherein the method comprises the use of MOF coated bioactive mitochondria.

28. A method of enabling the intracellular and in vivo delivery of bioactive mitochondria, the method comprising coating the isolated bioactive mitochondria with non-toxic Metal Organic Frameworks (MOFs) and transfecting the cells with the MOF coated bioactive mitochondria.

29. The method according to claim 28 for treatment of disorders associated with mitochondrial dysfunction, in particular for the treatment of cancer, metabolism related diseases, degenerative diseases, neoplastic diseases, neurodegenerative diseases, neuroimmune disorders, autoimmune diseases, tissue and organ regeneration and repair, aging, and mitochondrial related genetic diseases.
